(19) United States
(12) Patent Application Publication
(10) Pub. No.: US 2021/0187045 A1
(43) Pub. Date: Jun. 24, 2021

(54) AQUEOUS LIQUID EXTRACT OF SPIRULINA FOR PREVENTING OR TREATING LIPIDO-CARBOHYDRATE METABOLISM DISORDERS

(71) Applicant: ALGOSOURCE, SAINT-NAZAIRE (FR)

(72) Inventor: Olivier LEPINE, SAINT-NAZAIRE (FR)

(21) Appl. No.: 16/771,163

(22) PCT Filed: Dec. 11, 2018
(86) PCT No.: PCT/FR2018/053196

§ 371 Date: Jun. 9, 2020

(30) Foreign Application Priority Data
Dec. 12, 2017 (FR) 1762007

(51) Int. Cl. A61K 35/748; A61K 38/16; A61P 1/16

(52) U.S. Cl. CPC A61K 35/748; A61P 1/16; A61K 38/164

(57) ABSTRACT

Composition comprising an aqueous liquid extract of phycobiliproteins, containing phycocyanin, which is obtained from cyanobacteria or micro-algae and which has not been subjected to any drying step, said composition being used to prevent or treat lipido-carbohydrate metabolism disorders in humans or mammals. In particular, the composition can be used to prevent or treat non-alcoholic hepatic steatosis or steatohepatitis.

### FIELD OF THE INVENTION

The present invention relates to the field of lipido-carbohydrate metabolism disorders, and relates more particularly to a composition usable in preventing or treating nonalcoholic hepatic steatosis.

### PRIOR ART

Nonalcoholic hepatic steatosis is a disease associated with considerable accumulation of lipids in the liver, and is notably defined by an accumulation above 5% of neutral lipids in the hepatocytes. This pathology represents a major public health problem as it currently affects 20 to 30% of the population of Western countries and may progress to steatohepatitis (NASH), or may even develop into nonalcoholic cirrhosis or else hepato-carcinoma. All of these pathologies are grouped together under the term NAFLD (Non Alcoholic Fatty Liver Disease). Unfortunately, no treatment is available at present for treating these pathologies and the only way of detecting them is to perform a liver biopsy. That is why research is now turning toward means for preventing the development of hepatic steatosis.

In the course of their research, the inventors discovered that a composition based on phycocyanin, and more particularly derived from a specific extract from microalgae or cyanobacteria such as Spirulina, has interesting properties with respect to these lipido-carbohydrate metabolism disorders, and notably can be used in the treatment or prevention of nonalcoholic hepatic steatosis.

In fact, all the studies in the prior art were carried out using either an extract obtained from dried Spirulina, or an extract obtained from fresh Spirulina, but the extracted product underwent a subsequent drying step (for example for dry storage).

### AIMS AND SUMMARY OF THE INVENTION

For this purpose, the present invention relates to a composition comprising an aqueous liquid extract of phycobiliproteins, containing phycocyanin, obtained from cyanobacteria or microalgae, without undergoing a drying step, used for preventing or treating lipido-carbohydrate metabolism disorders in humans or other mammals. The composition according to the invention is used for protecting the liver against accumulation of lipids in the hepatocytes, and more particularly for preventing or treating nonalcoholic hepatic steatosis or steatohepatitis.

The method for preparing this extract, carried out in an aqueous medium only (without organic solvent), is described in detail in the patent application (not yet published) FR 1752452.

Not having undergone any drying step during its preparation, this extract notably contains a phycocyanin that is neither denatured nor degraded.

Preferably, the aqueous extract is obtained from Spirulina platensis.

Preferably, the composition according to the invention contains only said aqueous extract, comprising a phycocyanin content of at least 0.5 g/L, preferably between 0.8 and 10 g/L, more preferably between 1 g/L and 5 g/L.

Advantageously, it is in liquid form and is formulated for administration by the oral route. Its main advantage is that it can be added for example to drinking water, in the form of a food supplement.

The results obtained to date indicate that dosages for preventing nonalcoholic hepatic steatosis by means of the composition according to the present invention are, surprisingly, advantageously between 0.1 and 10 mg/kg of body weight/day of phycocyanin, preferably between 0.5 and 5 mg/kg/day of phycocyanin, i.e. at doses of phycocyanin that is neither denatured nor degraded that are well below the doses used in the prior art with dried Spirulina or dry extracts. This makes it possible to envisage applications that are more advantageous, notably in terms of economics.

At higher doses, the composition could be used for treating nonalcoholic steatosis.

### BRIEF DESCRIPTION OF THE DRAWINGS

The invention will become clear on reading the following description of embodiment examples, referring to the appended drawings, in which:

FIG. 1 is a flow chart of the protocol used for hamsters according to example 1, including a composition according to the invention.

FIG. 2 shows the parameters measured in the study according to the protocol in FIG. 1:
A: Fasting glycemia
B: Hepatic cholesterol content (mg/g of liver)
C: Aortic cholesterol content (mg/g of aorta)

FIGS. 3 to 18 relate to example 2 according to the invention:

FIG. 3 shows the food consumption of the mice at 15 weeks of treatment:
A: Water intake (ml/mouse/day)
B: Food intake (g/mouse/day)

FIG. 4 is a diagram showing the consumption of phycocyanin at 15 weeks of treatment in mg/mouse/day

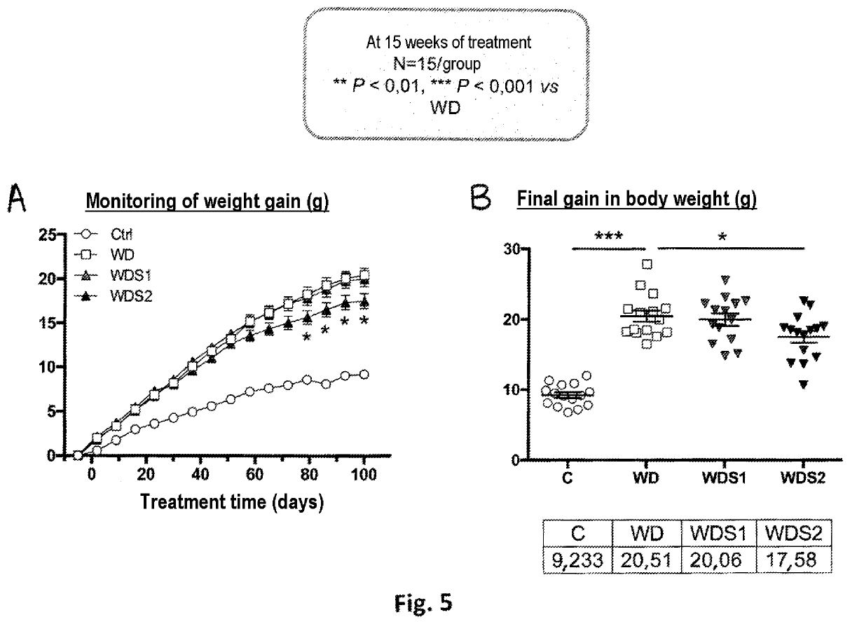

FIG. 5 shows monitoring of body weight:
A: as a function of treatment time in days, monitoring of weight gain in grams
B: final gain in body weight (in g) at 15 weeks of treatment

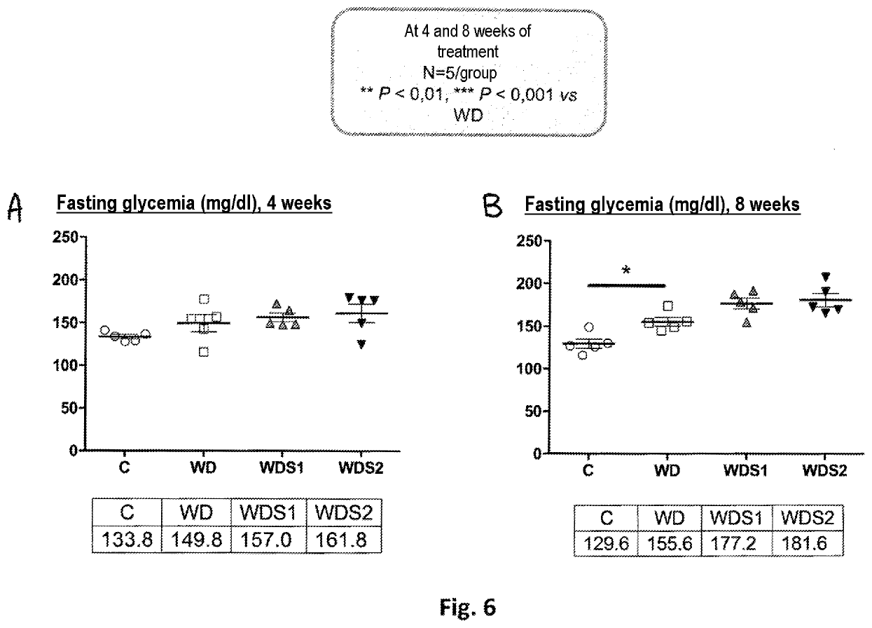

FIG. 6 shows fasting glycemia (mg/dl):
A: at 4 weeks
B: at 8 weeks

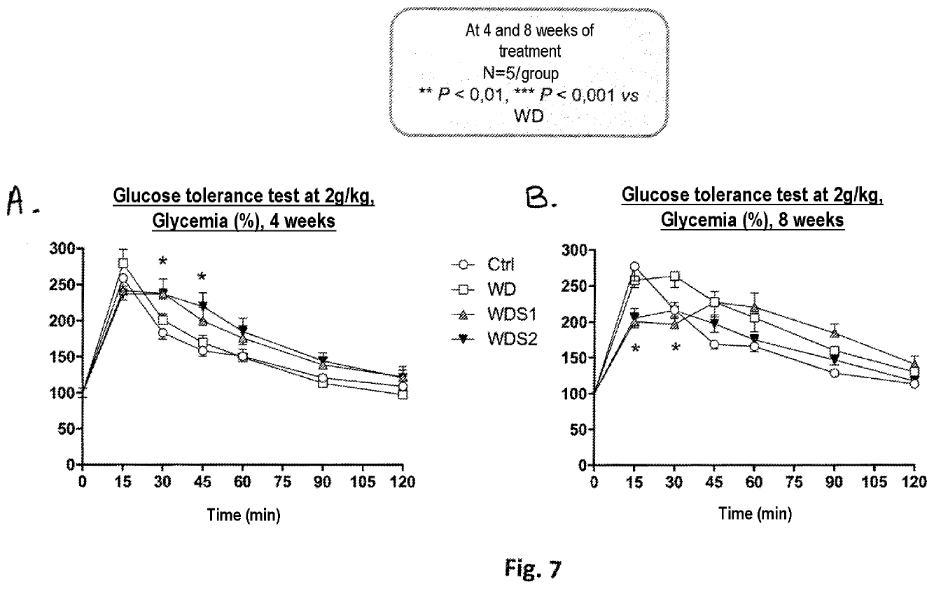

FIG. 7 shows tests of glucose tolerance at 2 g/kg, i.e. glycemia in %:
A: at 4 weeks
B: at 8 weeks

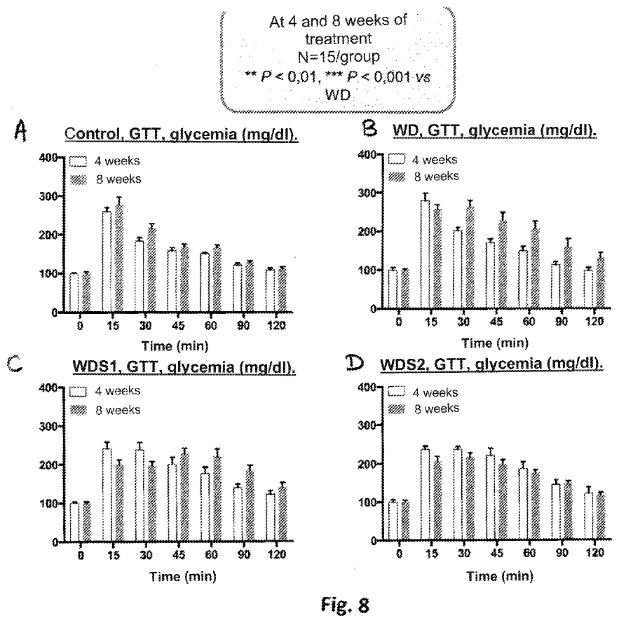

FIG. 8 shows other metabolic measurements at 4 and 8 weeks of treatment (glucose tolerance test GTT, and glycemia in mg/dl):
A: Control group
B: WD
C: WDS1
D: WDS2

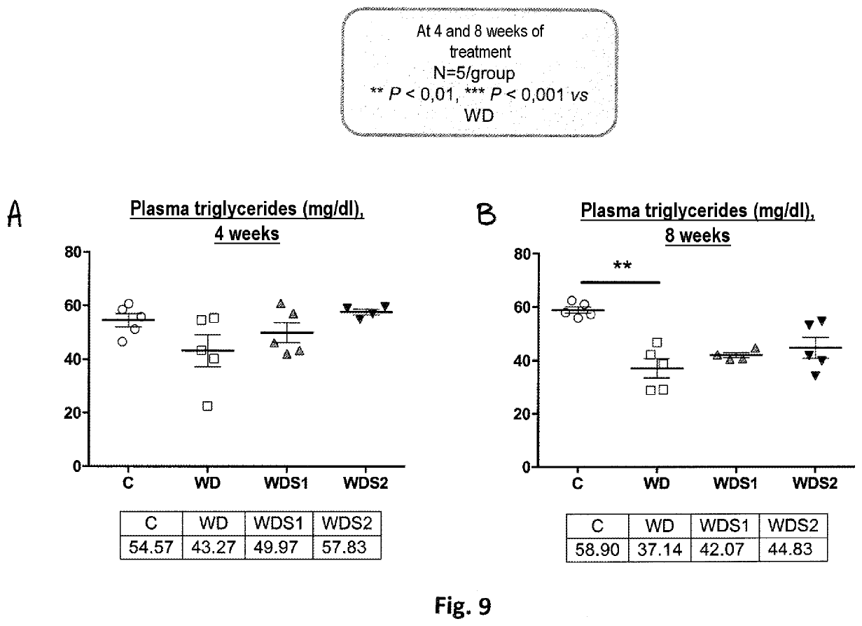

FIG. 9 shows plasma triglyceride concentrations (mg/dl):
A: at 4 weeks
B: at 8 weeks

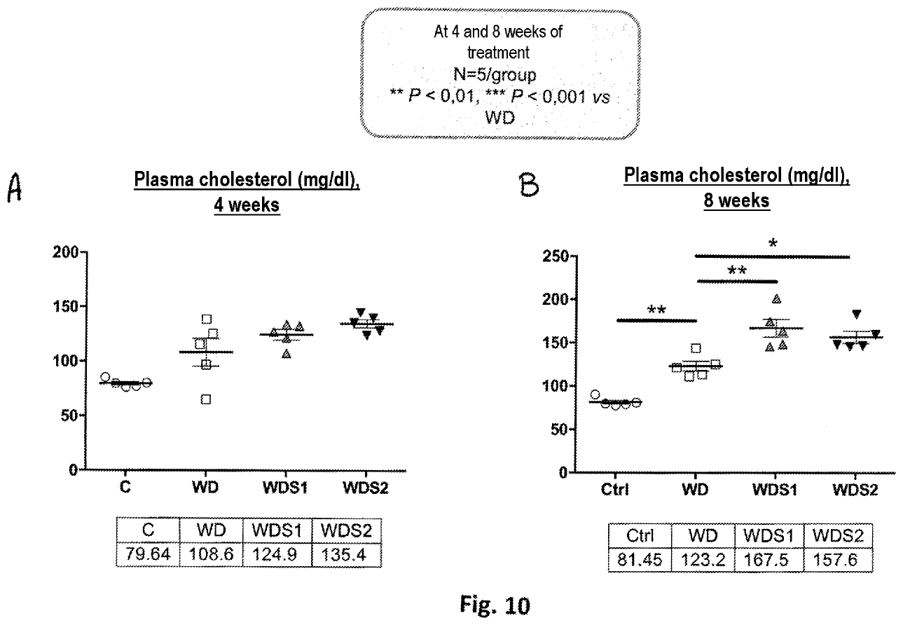

FIG. 10 shows other biochemical parameters, namely plasma cholesterol (mg/dl):
A: at 4 weeks
B: at 8 weeks

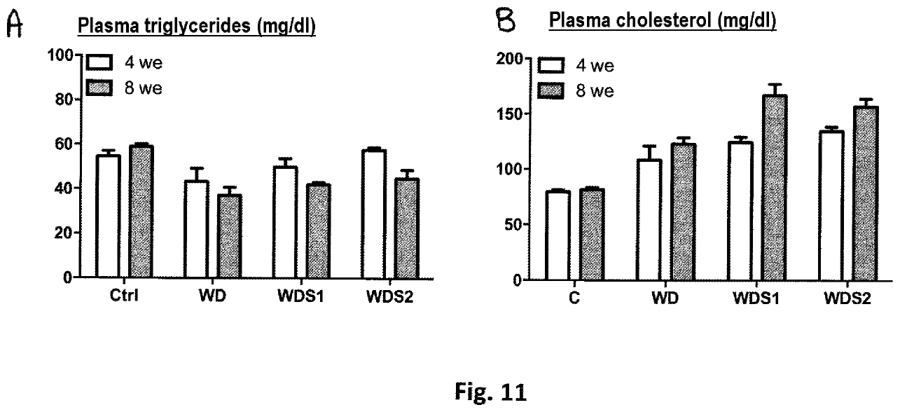

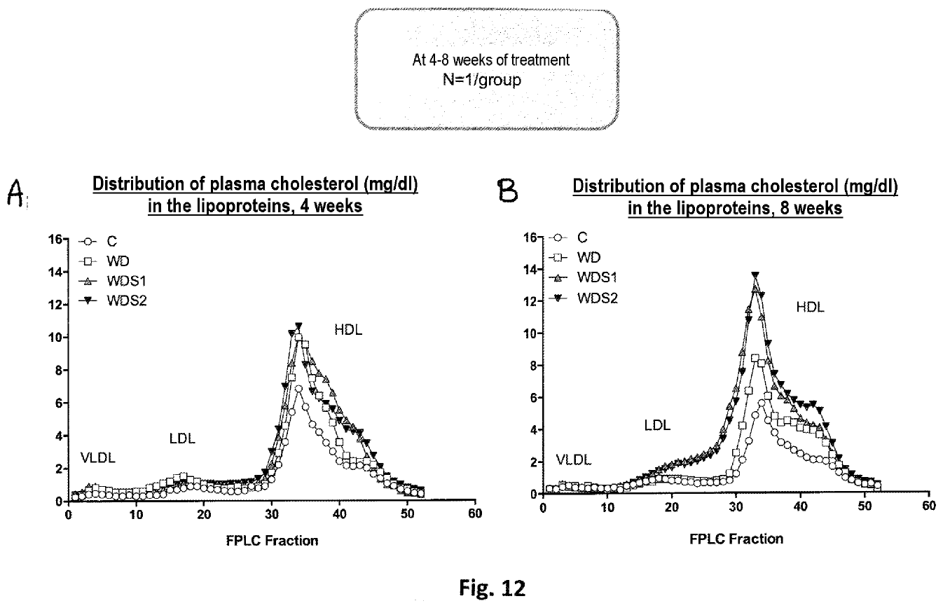

FIG. 11 is another presentation of the variation of the biochemical parameters compared to 4 and 8 weeks
A: plasma triglycerides
B: plasma cholesterol

FIG. 12 shows the distribution of plasma cholesterol (mg/dl) in the lipoproteins:
A: at 4 weeks
B: at 8 weeks

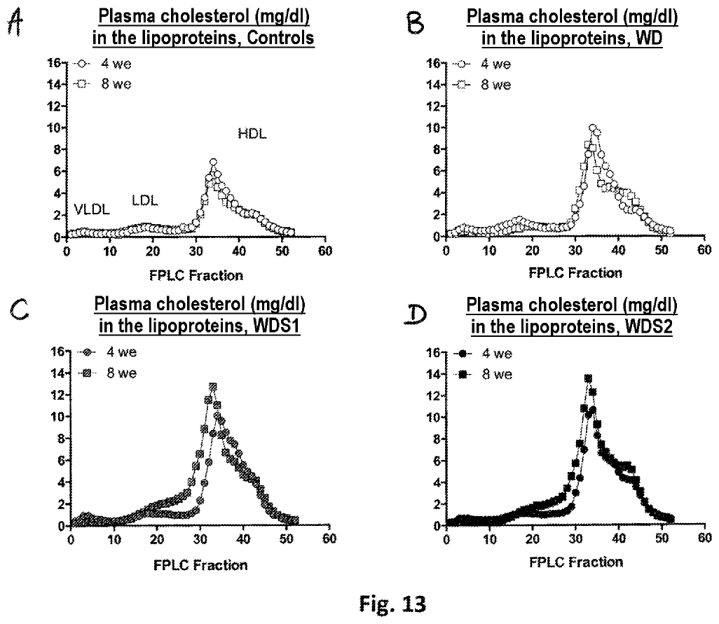

FIG. 13 gives details of the variation of plasma cholesterol in the lipoproteins for each of the groups:
A: Control group
B: WD
C: WDS1
D: WDS2

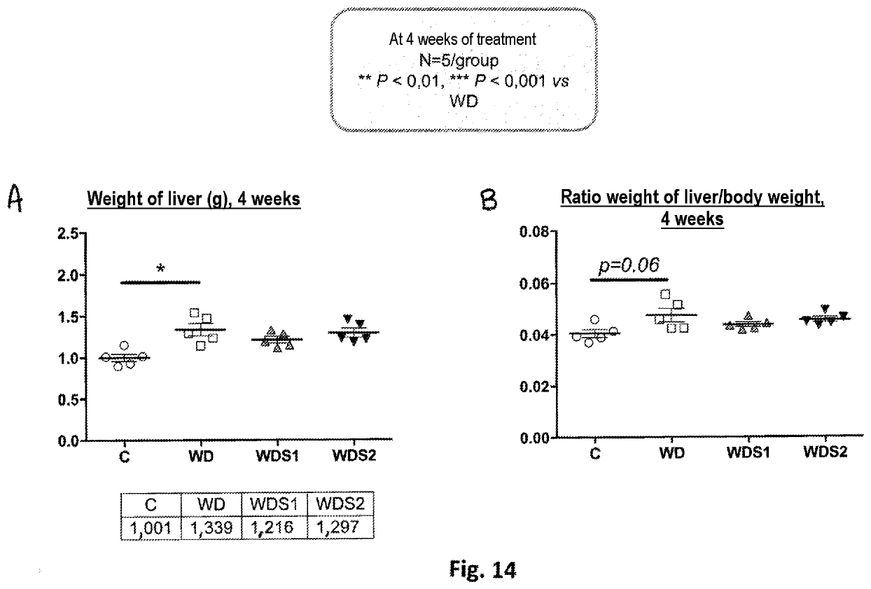

FIG. 14 shows liver analyses:
A: weight of the liver in grams at 4 weeks
B: ratio of weight of liver/body weight of the animal at 4 weeks

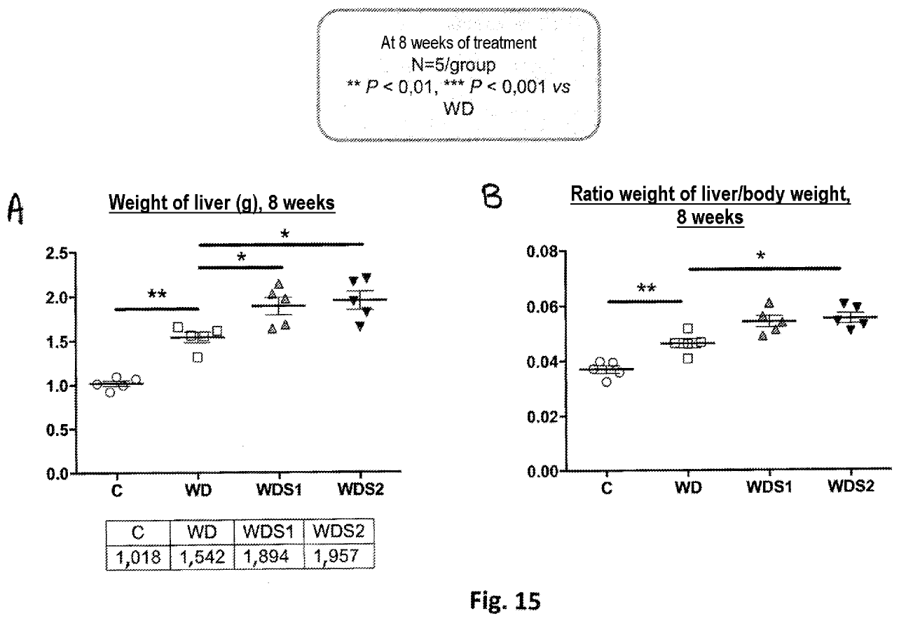

FIG. 15 shows the same parameters as FIG. 14 at 8 weeks:
A: weight of the liver in grams
B: ratio of weight of liver/body weight of the animal

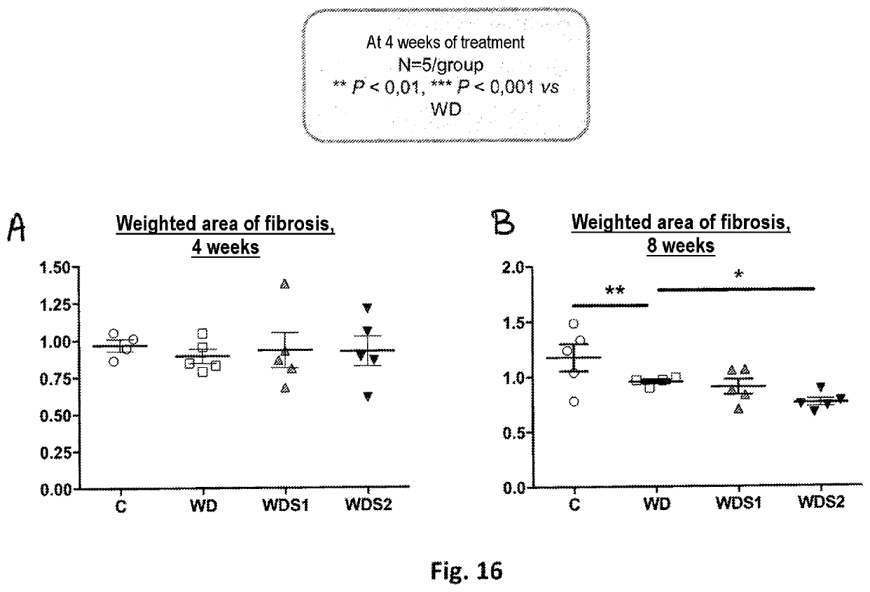

FIG. 16 concentrates on fibrosis:
A: Weighted area of fibrosis at 4 weeks
B: Weighted area of fibrosis at 8 weeks

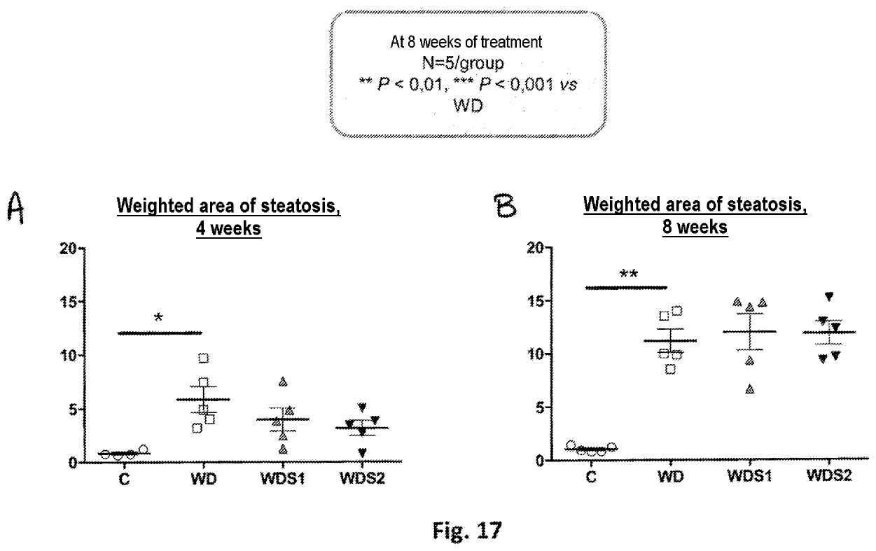

FIG. 17 shows measurements of steatosis:
A: Weighted area of steatosis at 4 weeks
B: Weighted area of steatosis at 8 weeks

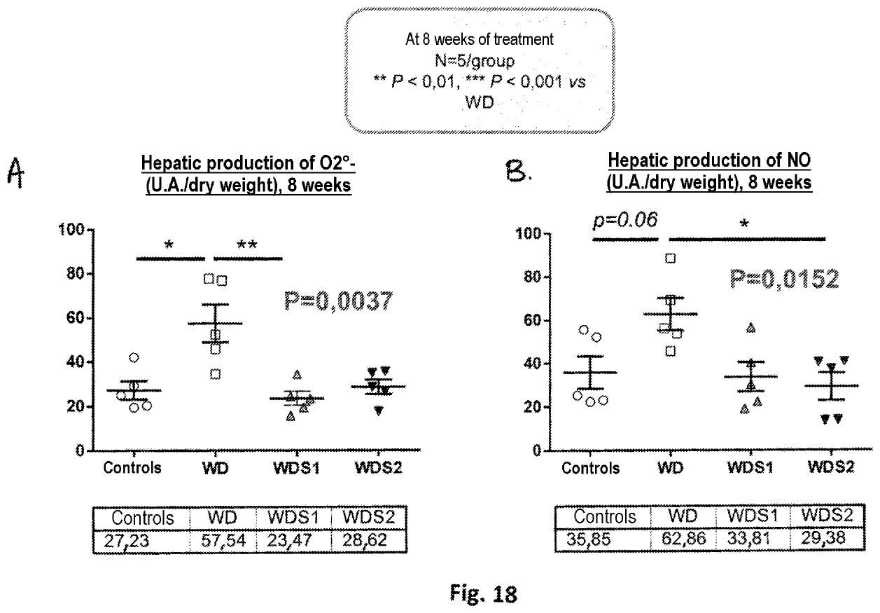

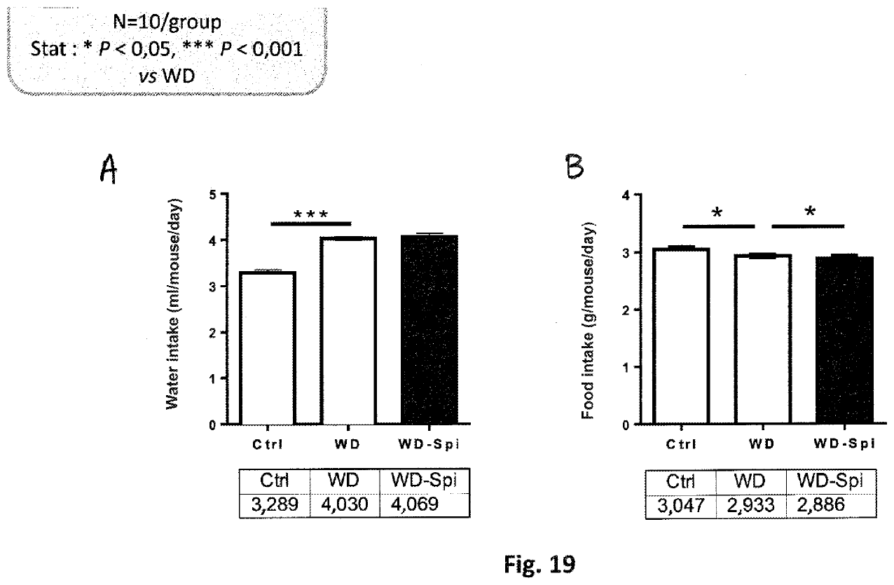

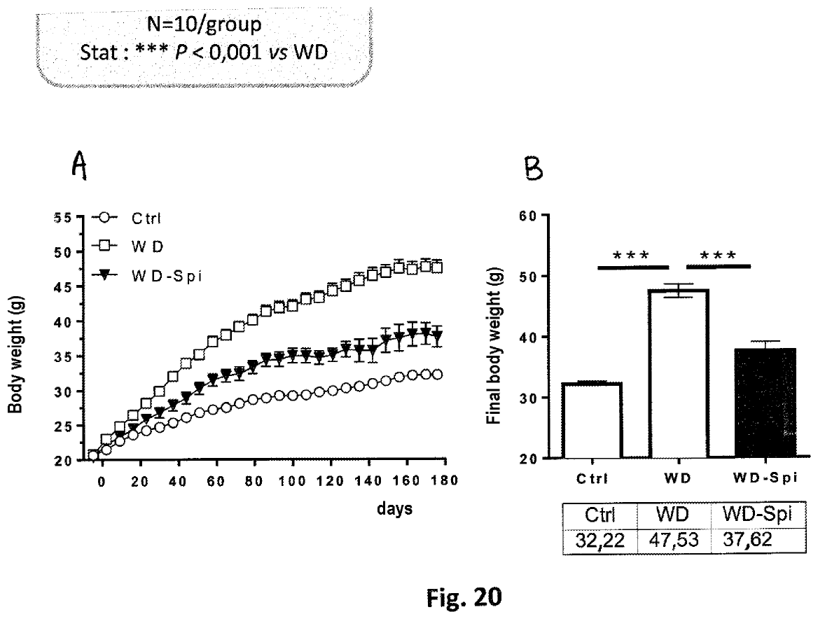

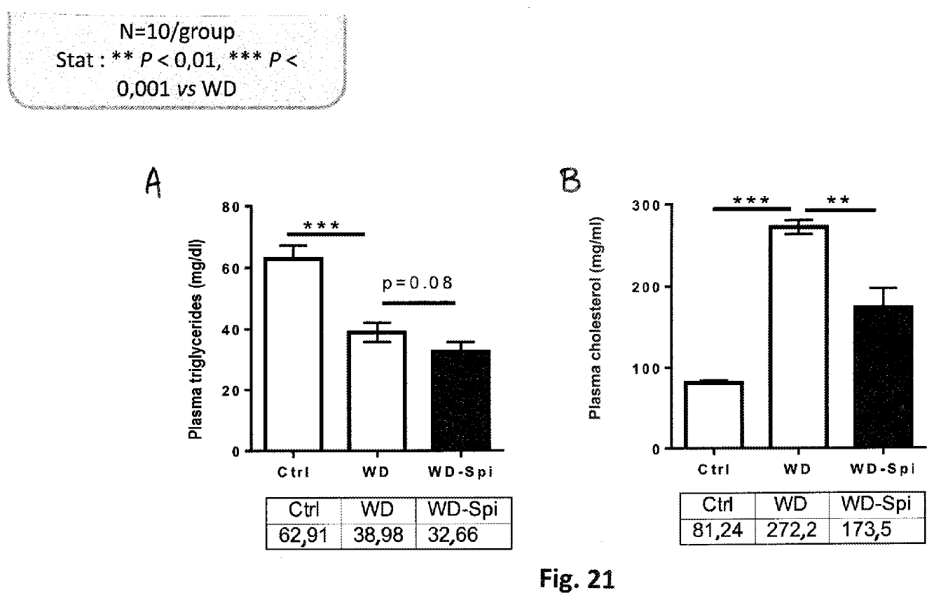

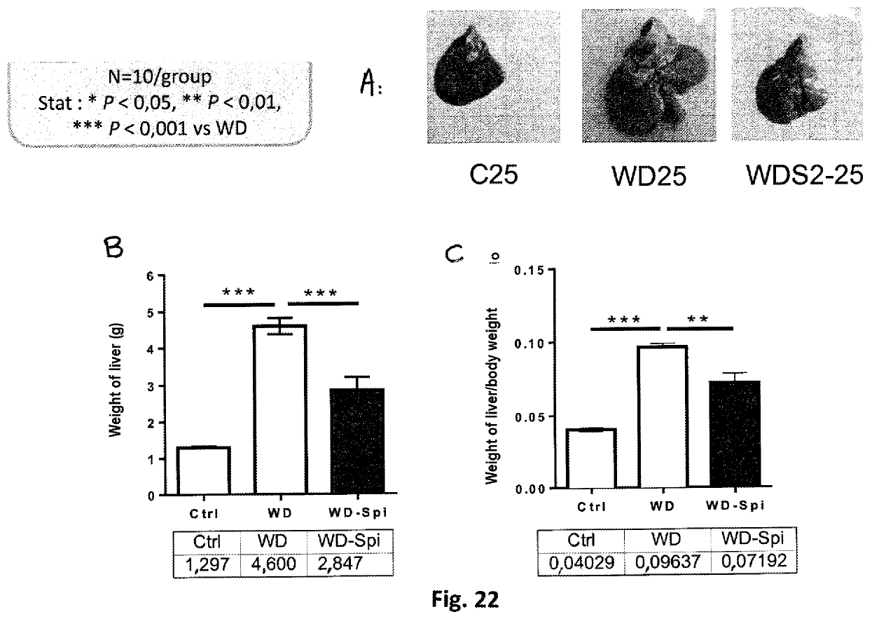

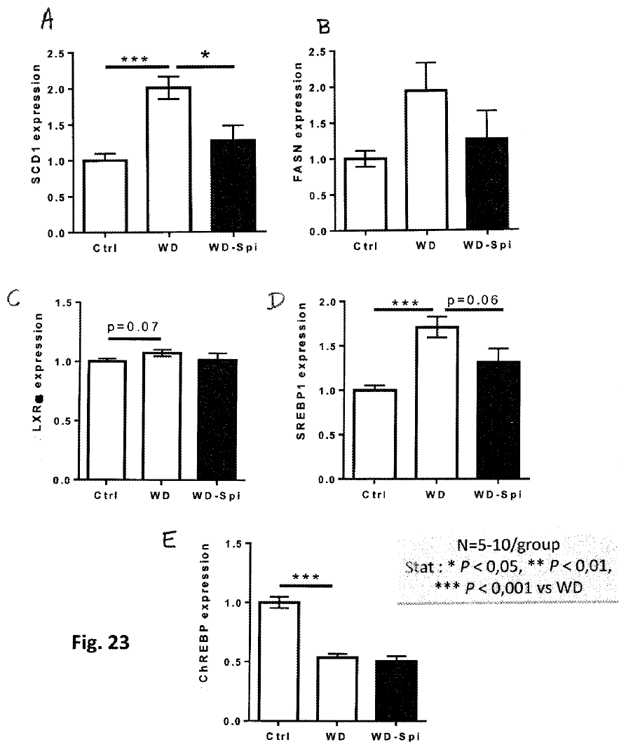

FIG. 18 shows measurements of oxidizing stress:
A: hepatic production of O2° at 8 weeks
B: hepatic production of NO at 8 weeks

FIGS. 19 to 23 relate to example 3 after 25 weeks of the diet

FIG. 19 shows the variation of food consumption:
A: water intake
B: food intake

FIG. 20 shows various measurements relating to weight:
A: variation of weight as a function of treatment time
B: a comparison of the final weight of the mice

FIG. 21 shows measurement of biochemical parameters at 25 weeks:
A: plasma triglycerides
B: plasma cholesterol

FIG. 22 shows measurements carried out on the liver:
A: photographs of the livers at the same scale, obtained after 25 weeks
B: weight of the liver
C: ratio of weight of liver/body weight

FIG. 23 shows the expression of different lipogenesis genes:
A: SCD1 expression
B: FASN expression
C: LXRα expression
D: SREBP1 expression
E: expression of ChREBP

### EXAMPLES

### Example 1

The effect of administration of phycocyanin was studied on hamsters, notably on their lipid metabolism. The phycocyanin was added to their food, more particularly in their drinking water, in the form of the liquid extract of Spirulina called Spirulysat®, marketed by the company Algosource. Spirulysat® is a liquid extract, the method of extraction of which (patent application FR1752452, not yet published), obtained from cyanobacteria called Arthrospira platensis, more commonly called Spirulina, which are very rich in antioxidant molecules such as phycocyanin (PC-C).

1.1 g of Spirulysat®/kg of body weight/day (i.e. 10 mg of phycocyanin/kg of body weight/day) was administered to four groups of hamsters:
a control group (C) fed with a normal diet,
a control group (CSp) fed with 10 mg of phycocyanin/kg of body weight/day,
a group fed a hyper-fat diet (HF), and
a group fed a hyper-fat diet supplemented with 80 mg of phycocyanin/kg of body weight/day (HFSp).

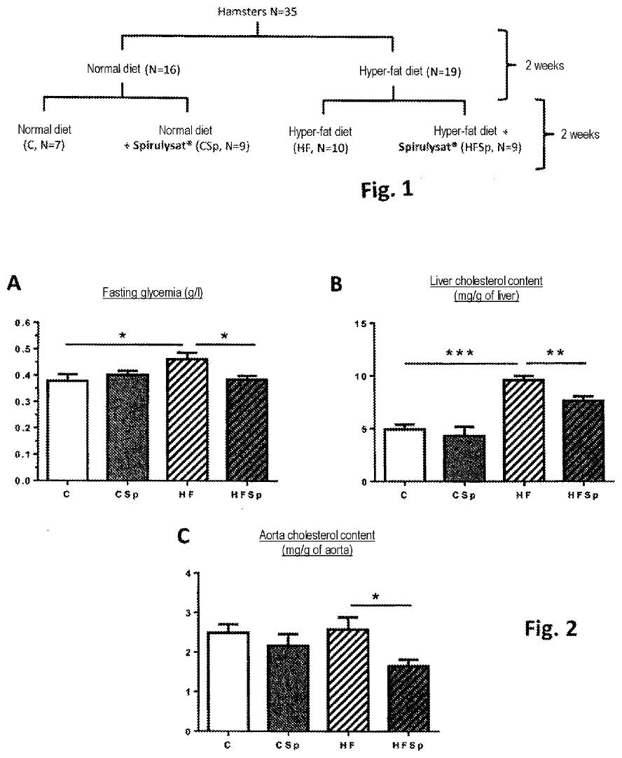

These hamsters had been fed a high-fat diet for 2 weeks beforehand to induce metabolic disturbances. At the end of this period, the hamsters continued to receive the same diet for two weeks in parallel with administration of Spirulysat® in the drinking water. The protocol applied is shown schematically in FIG. 1.

The key results of this study are defined essentially in 3 points. Firstly, a significant decrease of fasting glycemia was observed in group HFSp compared to group HF (−17%), without a change in weight of the hamsters (145.9±5.45 vs 142.1±4.48 g, respectively) (FIG. 2A). Secondly, a considerable decrease in liver cholesterol was noted in group HFSp relative to group HF (7.65±0.43 vs 9.61±0.41 mg of cholesterol/g of liver, respectively) (FIG. 2B). It can also be seen that there is a tendency for less hepatic accumulation of free fatty acids (14.94±0.71 vs 12.77±0.69 μmol/g of liver for HF and HFSp respectively, p=0.06) and triacylglycerols (4.95±0.39 vs 4.01±0.44 mg/g of liver for HF and HFSp, respectively, p=0.09). Finally, the hamsters that drink water supplemented with Spirulysat® accumulate less cholesterol at the level of the aorta, when they are challenged with a high-fat diet (2.58±0.31 vs 1.65±0.17 mg of cholesterol/g of aorta for groups HF and HFSp, respectively) (FIG. 2C).

### Example 2

Following the results obtained on the hamsters in example 1, a more complete study was carried out on C57Bl/6 mice. The C57Bl/6 mice are models for studying liver diseases, as they are sensitive to a hyper-fat diet and develop the risk factors for NAFLD.

This protocol comprised four groups of animals:
a control group (Ctrl) fed with a normal diet,
a group (WD) fed with a hyper-fat diet (23% of lipids and 2% of cholesterol) and containing fructose in the drinking water,
a group (WDS1) fed with a hyper-fat diet that contained fructose and Spirulysat® at 30 mg of phycocyanin/kg of body weight/day in the drinking water, and
a group (WDS2) fed with a hyper-fat diet that contained fructose and Spirulysat® at a dose 5 times higher, i.e. at 150 mg of phycocyanin/kg of body weight/day in the drinking water.

The NAFLDs include several diseases that correspond to the different stages of liver disease. The study is thus a kinetic study of the onset of these different stages.

For this purpose, 10 mice from each of the groups in the above protocol were sacrificed after 4, 8, 16, 25 and 35 weeks. The diet used and the duration of exposure to this diet were selected according to the work by Charlton, in American Journal of Physiology-Gastrointestinal and Liver Physiology, 2011, 301(5): G825-34, showing similarity in the pathophysiological development of NAFLDs between mice and humans.

2.1. Food Consumption and Body Weight

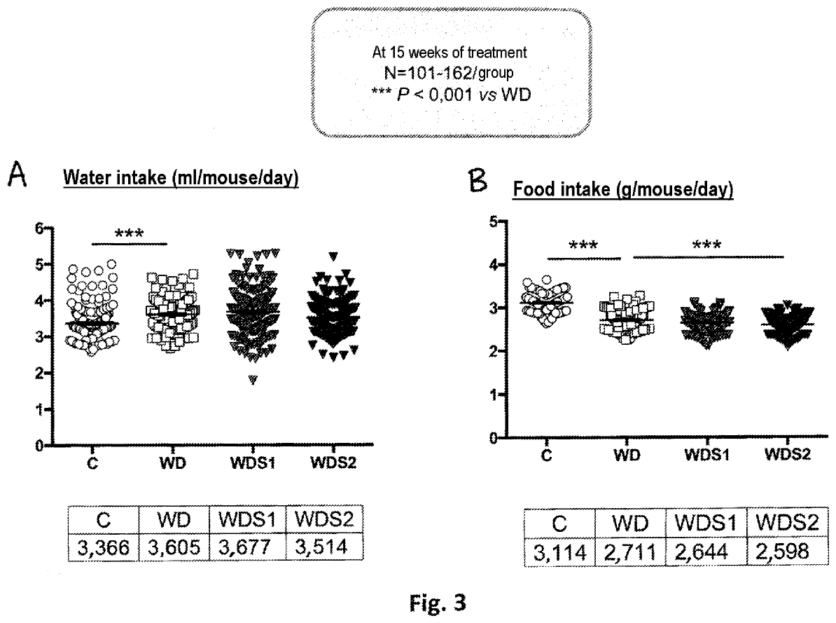

The results show that the mice WD consume significantly less hyper-fat pellets than the mice with the reference diet (group Ctrl) (12%, see FIG. 3B). In fact, the hyper-fat diet, called “western”, supplies 42% more calories than the control diet for identical weight of pellets. Moreover, this is associated with an increase in the consumption of drinking water containing fructose (3.45±0.04 vs 3.28±0.03 ml/mouse/day respectively) (FIG. 3A), meaning a daily calorie intake about 30% higher in group WD compared to group Ctrl. However, it is noted that the presence of Spirulysat® in the food for the mice does not alter their water consumption, but decreases their food intake.

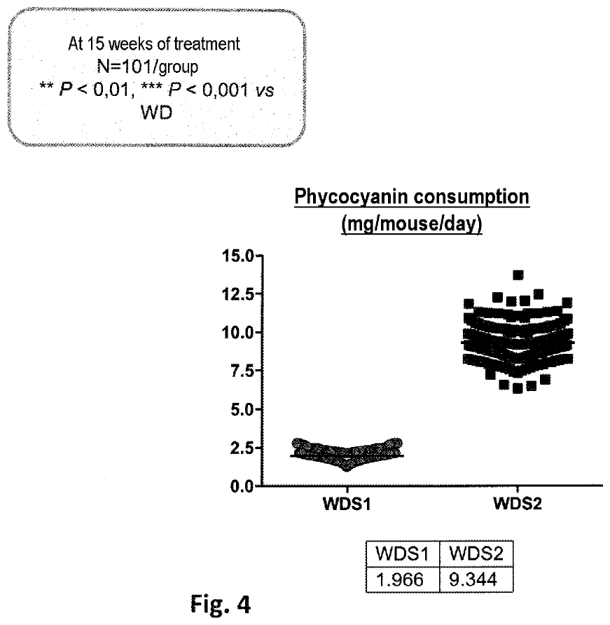

FIG. 4 shows that the consumption of phycocyanin is about 5 times higher in the mice in group WDS2 than in the mice in group WDS1.

Monitoring the weight of the animals reveals that the hyper-fat diet certainly plays a part, since the mice in WD all have a significantly higher body weight than the Ctrl mice (FIGS. 5A and B). After 15 weeks of the diet, the body weight is less in the mice that ingested Spirulysat®.

2.2 Metabolic Measurements

The measurements of glycemia show that ingestion of Spirulysat® does not decrease fasting glycemia (FIGS. 6A and B and FIG. 8), but does decrease glycemic excursion (FIGS. 7A and B) starting from 4 and 8 weeks of diet, respectively.

2.3 Biochemical Parameters

There is no notable effect of food containing Spirulysat® on plasma triglycerides (FIGS. 9A and B and FIG. 11A), but there is an increase in plasma cholesterol (FIGS. 10A and B and FIG. 11B).

Investigation of the distribution of this plasma cholesterol shows that the concentration of cholesterol in HDLs (called “good cholesterol”) is clearly increased in the mice whose food contains Spirulysat® (see the various diagrams in FIGS. 12 and 13).

2.4 Liver Analyses

Steatosis and liver fibrosis were quantified respectively after trichrome staining by adding saffron and staining with 0.1% picrosirius red.

Comparing FIGS. 14 and 15, it can be seen that in groups WDS1 and WDS2, the weight of the liver has increased at 8 weeks. However, at 8 weeks of the diet, the weighted area of fibrosis is definitely reduced in the WDS2 group of mice (400 mg of phycocyanin); the steatosis values are not significant at 8 weeks (FIG. 17).

Hepatic OS (oxidizing stress) (O2°—and NO) was measured by electron paramagnetic resonance.

Oxidizing stress, induced by the hyper-fat diet with fructose (group WD), is greatly reduced in the mice that received phycocyanin WDS1 and WDS2, and is brought back to values equivalent to those of the control group (Ctrl), as shown by the values in FIG. 18, diagrams A (hepatic production of O2°) and B (hepatic production of NO).

### Example 3

The experiments according to example 2 were conducted for 25 weeks on three groups of C57Bl/6 mice aged 7 weeks:
a control group (Ctrl) fed with a normal diet,
a group (WD) fed with a hyper-fat diet (23% of lipids and 2% of cholesterol) and containing 42 g/l of fructose in the drinking water, and
a group (WD-Spi) fed with a hyper-fat diet (23%) and containing 42 g/l of fructose and Spirulysat® at 150 mg of PC-C/kg of body weight/day in the drinking water.

The results obtained notably confirm that the liquid extract of Spirulina, at 25 weeks of the diet:
decreases food intake (FIG. 19B)
decreases body weight and the gain in fat mass (FIG. 20)
decreases plasma triglycerides and total cholesterol (FIG. 21)
reduces the increase in weight of the liver relative to that of group WD (FIG. 22)
and finally modulates the expression of the genes involved in the mechanisms of antioxidant defense and lipogenesis (FIG. 23).

1. A composition comprising:
an aqueous liquid extract of phycobiliproteins, containing phycocyanin, obtained from cyanobacteria or microalgae, without undergoing a drying step, used for protection of the liver against accumulation of lipids in the hepatocytes.

2. The composition as claimed in claim 1, wherein said composition is used for preventing or treating nonalcoholic hepatic steatosis or steatohepatitis.

3. The composition as claimed in claim 1, wherein the aqueous extract is obtained from Spirulina platensis.

4. The composition as claimed in claim 1, wherein said composition contains only said aqueous extract, comprising a phycocyanin content of at least 0.5 g/L, preferably between 0.8 g/L and 10 g/L, more preferably between 1 g/L and 5 g/L.

5. The composition as claimed in claim 1, wherein said composition is in liquid form and is formulated for administration by the oral route.

6. The composition as claimed in claim 1, wherein said composition is used at preventive dosages between 0.1 and 10 mg/kg of body weight/day of phycocyanin.

7. The composition as claimed in claim 1, wherein said composition is used at preventive dosages between 0.5 and 5 mg/kg of body weight/day of phycocyanin.
